(19) United States
(12) Patent Application Publication
(10) Pub. No.: US 2022/0381021 A1
(43) Pub. Date: Dec. 1, 2022

(54) Analytical Toilet with Microfluidic Chip

(71) Applicant: Medic, Inc., Provo, UT (US)

(72) Inventor: K. Jeffrey Campbell, Spanish Fork, UT (US)

(73) Assignee: Medic, Inc., Provo, UT (US)

(21) Appl. No.: 17/686,402

(22) Filed: Mar. 3, 2022

Related U.S. Application Data
(60) Provisional application No. 63/155,978, filed on Mar. 3, 2021.

(51) Int. Cl. E03D 9/00; B01L 3/00; G01N 21/25

(52) U.S. Cl. CPC E03D 9/00; B01L 3/502715; G01N 21/25; B01L 2200/04; B01L 2200/027; B01L 2200/16; B01L 2400/0622; B01L 2300/0663

(57) ABSTRACT

An analytical toilet is disclosed with a bowl adapted to receive excreta, a conduit for transporting a liquid excreta sample from the bowl, and a liquid reagent source. The analytical toilet also includes a microfluidic chip that has a sensor configured to detect at least one property of the excreta sample. The microfluidic chip also has an excreta sample path in fluid communication with the conduit and the sensor and a reagent path in fluid communication with the liquid reagent source and the sensor. The length of and number of channels in the sample path and the reagent path are selected so as to control the respective fluid resistance of the excreta sample and the reagent to thereby optimize the mixing and flow rates of the excreta sample and reagent into the sensor. There is also disclosed analytical toilet with a microfluidic chip having reagent path that includes a first and a second channel. The second channel is longer than the first channel. A valve, which is controllable so as to cause the reagent to flow through either the first channel, the second channel or both channels. As such, the fluid resistance of the reagent is controlled, to thereby optimize the flow rate of the reagent into the sensor.

### CROSS-REFERENCE TO RELATED APPLICATION

This application is a continuation-in-part of U.S. Provisional Patent Application No. 63/155,978, filed Mar. 3, 2021 and entitled Variable Resistance Microfluidic Chip for Analytical Toilet, the entire disclosure of which is incorporated herein by this reference.

### TECHNICAL FIELD

The present disclosure relates to analytical toilets. More particularly, it relates to analytical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

An analytical toilet performing health and wellness testing on excreta requires special plumbing to and from sensors for excreta, reagents, diluent, and flush water among others. Because there is limited space within the toilet for the sensors and necessary plumbing, it would be advantageous for a single sensor to be able to detect a variety of analytes in excreta. A microchannel chip for continuously diluting a solution and dilution method are disclosed in KR 2007/0035440 the entire disclosure of which is incorporated herein by reference.

Just a few examples of smart toilets and other bathroom devices can be seen in the following U.S. patents and Published applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; and US 2018/0165417, entitled “Bathroom Telemedicine Station.” The disclosures of all these patents and applications are incorporated by reference in their entireties.

### SUMMARY

In a first aspect, the invention is an analytical toilet with a bowl adapted to receive excreta, a conduit for transporting a liquid excreta sample from the bowl, and a liquid reagent source. The analytical toilet also includes a microfluidic chip that has a sensor configured to detect at least one property of the excreta sample. The microfluidic chip also has an excreta sample path in fluid communication with the conduit and the sensor and a reagent path in fluid communication with the liquid reagent source and the sensor. The length of and number of channels in the sample path and the reagent path are selected so as to control the respective fluid resistance of the excreta sample and the reagent to thereby optimize the mixing and flow rates of the excreta sample and reagent into the sensor.

Preferably, in this first aspect, the there is also provided a source of diluent and a diluent path in the microfluidic, which is also tailored for optimum flow and mixing.

Preferably, in this first aspect, the length of the sample path is at least twice as long as the reagent path, to thereby provide more reagent to the sensor than excreta sample under the sample pressure. Alternatively, wherein the reagent path comprises two or more channels, to thereby provide more reagent to the sensor than excreta sample under the same pressure.

Preferably, in this first aspect, the microfluidic chip further includes an outlet in fluid communication with the sensor.

In this first aspect, the reagent source and microfluidic chip are preferably contained on a cartridge configured to fit into a receptacle in the toilet.

In a second aspect, the invention is an analytical toilet with a bowl adapted to receive excreta, a conduit for transporting a liquid excreta sample from the bowl, and a liquid reagent source. The analytical toilet also includes a microfluidic chip having a sensor configured to detect at least one property of the excreta sample, an excreta sample path in fluid communication with the conduit and the sensor, and a reagent path in fluid communication with the liquid reagent source and the sensor. The reagent path includes a first and a second channel, wherein the second channel is longer than the first channel. The microfluidic chip also includes a valve, which is controllable so as to cause the reagent to flow through either the first channel, the second channel or both channels. As such, the fluid resistance of the reagent is controlled, to thereby optimize the flow rate of the reagent into the sensor.

Preferably, in this second aspect, the valve is controllable to cause the reagent to flow through either the first channel or the second channel, but not both, to thereby change the length of the reagent path and thereby increase the fluid resistance of resistance of the reagent path when the second channel is selected.

Alternatively, the valve is controllable to cause the reagent to flow through either the first channel or both the first and the second channel simultaneously, to thereby decrease the fluid resistance when the reagent flow through both the first and second channel simultaneously.

In this second aspect, the reagent source may be filled with either a first or a second reagent, depending on what analysis is to be performed, and the valve is controlled depending on whether the first or the second reagent is in the reagent source.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a perspective view of a first exemplary embodiment of an analytical toilet according to the present disclosure.

FIG. 2 is a rear perspective view of the toilet of FIG. 1 with the rear compartment open.

FIG. 3 is a side perspective view of the toilet of FIG. 1 with a side panel removed to show the interior of the toilet.

FIG. 4 is a side perspective view of the manifold of the toilet of FIG. 1.

FIG. 5 is a perspective view of a first exemplary embodiment of an analytical test device attached to a medical toilet according to the present disclosure.

FIG. 6 is an exploded perspective view of an exemplary embodiment of a microfluidic lab on chip fluid interface.

FIG. 7 is an exploded perspective view of an exemplary embodiment of an optical interface for a microfluidic system, in lateral configuration.

FIG. 8 is a schematic view of an exemplary embodiment of a microfluidic chip according to the present disclosure.

FIG. 8A is a closeup view of a portion of FIG. 8 marked as “A”.

FIG. 9 is a schematic view of another exemplary view of a microfluidic chip according to the present disclosure.

FIG. 10 is a schematic view of yet another exemplary view of a microfluidic chip according to the present disclosure.

FIG. 11 is a schematic view of still yet another exemplary view of a microfluidic chip according to the present disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, xray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, the term “microfluidics” is meant to refer to the manipulation of fluids that are contained to small scale, typically sub-millimeter channels. The prefix “micro” used with this term and others in describing this invention is not intended to set a maximum or a minimum size for the channels or volumes.

As used herein, the prefix “nano” is meant to refer to something in size such that units are often converted to the nano-scale for ease before a value is provided. For example, the dimensions of a molecule may be given in nanometers rather than in meters.

As used herein, “bind” and similar variants are meant to refer to the property of facilitating molecular interaction with a molecule, such as interaction with a molecular biomarker.

### Exemplary Embodiments

The present disclosure relates to analytical toilets (may also be referred to as an “analytical toilet” or a “health and wellness” toilet) with analytical tools to perform scientific tests on excreta samples to identify potential health and wellness indicators. More particularly, it relates to the use of modular testing devices with standardized mechanical connection and interfaces providing, as appropriate for particular tests, electrical power, data connection, fluid inlets, and fluid outlets.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

In various exemplary embodiments, the analytical toilet provides a fluid processing manifold that collects and routes samples from the toilet bowl to various scientific test devices and waste handling portals throughout the device. In a preferred embodiment, the manifold is digitally controlled. A digitally controlled manifold may include analog component or circuits.

In various exemplary embodiments, the medical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, buffer solutions, reagents, water, cleaners, biomarkers, dilution solutions, calibration solutions, and air. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive all common supplies (e.g., excreta samples, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different test methods and to test excreta samples for different constituents.

In various exemplary embodiment, the smart toilet provides an electrical power connection and a data connection for the analytical test device. In a preferred embodiment, the electrical power and data connections use the same circuit. In various exemplary embodiments, the toilet is provided with pneumatic and/or hydraulic power to accommodate the analytical test devices.

In various exemplary embodiments, the smart toilet platform performs various functions necessary to prepare samples for examination. These functions include, but are not limited to, liquidizing fecal samples, diluting or concentrating samples, large particle filtration, sample agitation, and adding reagents. Miniaturized mechanical emulsification chambers show promise for repeatable and sanitary stool preparation. Stool samples may also be liquefied using acoustic energy and/or pressurized water jets.

In various exemplary embodiments, the smart toilet also provides, among other things, fluid transport, precise fluid metering, fluid valving, fluid mixing, separation, amplification, storage and release, heating of fluid, cooling of fluid, and incubation. The smart toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test devices, allowing bulk mixed sample to be routed to anywhere from one to all stations (i.e., analytical test device interfaces) for analysis. Mixing may also occur in an analytical test device.

In various exemplary embodiments, samples are filtered for large particulates at the fluid ingress ports of the manifold. The fluid manifold uses a network of horizontal and vertical channels along with simple valves to route prepared stool samples to one of several scientific test devices located on the platform.

In various exemplary embodiments, samples are filtered for particulates larger than a particular threshold, defined appropriately for the application. In some embodiments, multiple filters may be used sequentially or be available in parallel. One layer of the manifold stack may be dedicated as an interchangeable filter module, or a more monolithic purpose-build manifold may be the design of choice. The fluid manifold uses a network of horizontal and vertical channels along with simple valves to route filtered urine or stool samples to one of several scientific test devices located on the platform. The filter mechanism is placed in series with all other scientific test devices, allowing filtered sample to be routed to anywhere from one to all stations (i.e., analytical test device interfaces) for analysis. Filtering may also occur in an analytical test device.

In various exemplary embodiments, the manifold is constructed using additive layers, and different layers can be customized for particular applications. Standard ports and layouts are used for interfacing with external components, such as pressure sources and flow sensors. In general, characteristic channel volumes at the first layers of the manifold stack are on the order of milliliters. At the final level of the manifold stack is the microfluidic science device, which will interface simultaneously with multiple microfluidic chips using standardized layout and pressure seals.

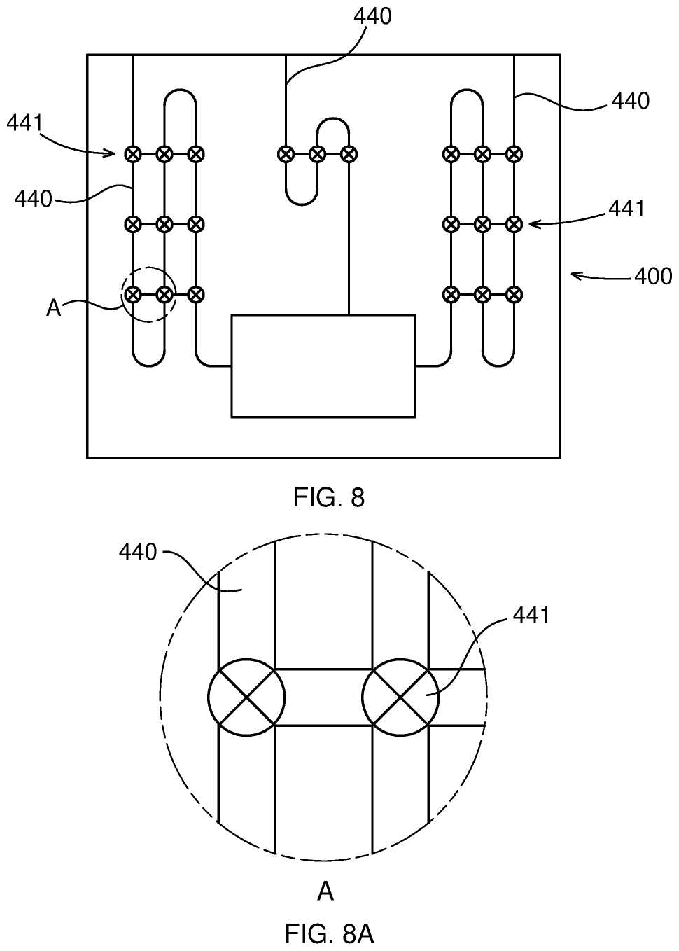

In accordance with the present invention, the analytical toilet is provided with at least one microfluidic chip. In order to achieve optimum mixing and flow rates for the fluids being processed on the microfluidic chip, the fluid resistance is manipulated by selecting the length and or number of channels for the path of the sample, the reagent, and optionally the diluent and second reagent. These manipulations are discussed in more detail in connection with FIGS. 8-11. In general, increasing the length of the channel will increase the fluid resistance. In turn, increasing the fluid resistance will decrease the flow rate of that liquid, assuming a constant pressure. Also, increasing the number of channels decreases the fluid resistance and thus increase the flow rate of that liquid.

In various exemplary embodiments, the analytic test devices are designed to perform one or more of a variety of laboratory tests in a toilet environment. Any test that could be performed in a medical or laboratory setting may be implemented in an analytical test device in a toilet. These tests may include measuring pulse, blood pressure, blood oxygenation, electrocardiography, body temperature, body weight, excreta content, excreta weight, excreta volume, excreta temperature, excreta density, excreta flow rate, and other health and wellness indicators.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfluidic actuation technologies including, but not limited to, pneumatic driven, mechanical pumps (e.g., peristaltic, diaphragm), on-chip check-valve actuators (e.g., piezo-driven or magnetic), electroosmotic driven flow, vacuum pumps, and capillary or gravity driven flow (i.e., with open channels and vents).

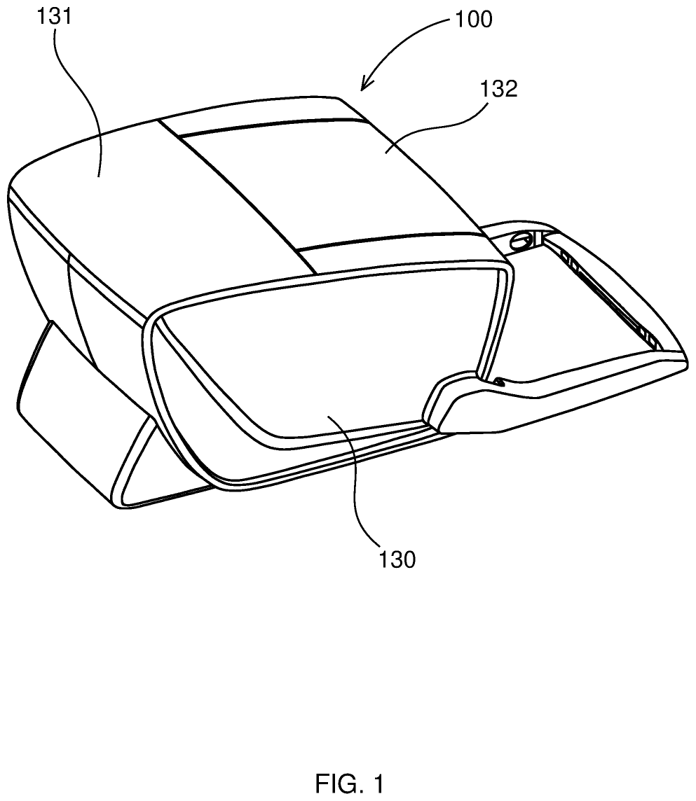

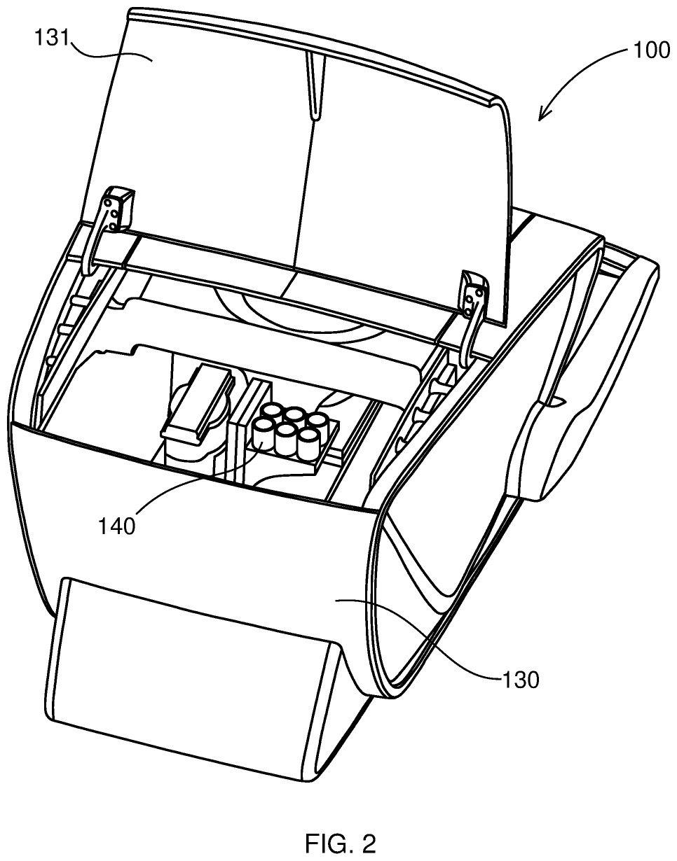

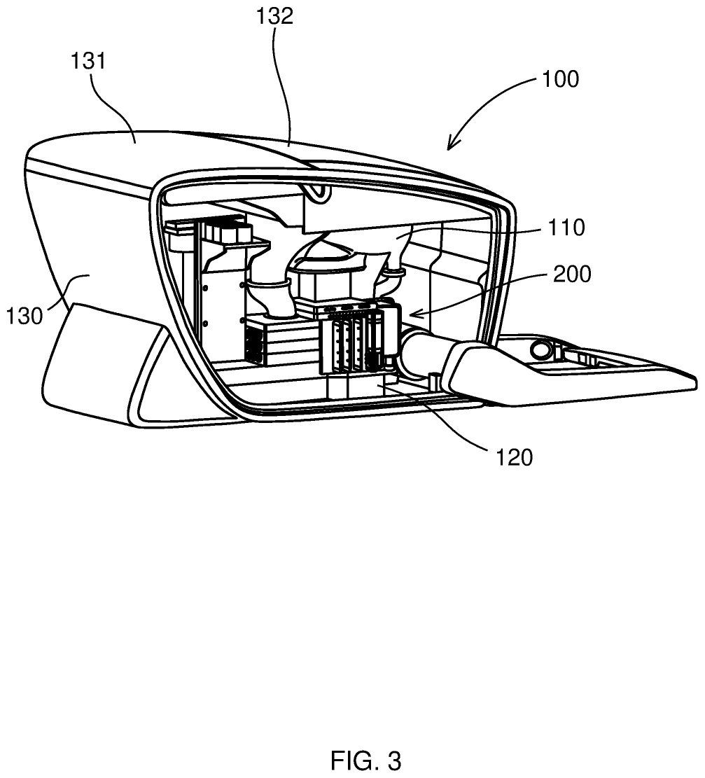

Now referring to FIGS. 1-3, a first embodiment of an analytical toilet 100 is shown. FIG. 1 shows the toilet seat, bowl, plumbing, and other internal components covered by a shroud 130 that includes a rear cover 131 and lid 132. FIG. 2 shows the toilet with a rear cover 131 of the shroud 130 open showing part of the interior of the toilet. In this embodiment, this portion of the toilet 100 includes fluid containers 140 that hold supplies used for some of the functions of the analytical toilet 100 (e.g., analytical tests, cleaning, disinfecting, sample preparation, etc.). FIG. 3 shows the toilet 100 with a side panel of the shroud 130 removed to allow showing the interior components of the toilet 100, including the bowl 110, base 120, and manifold 200.

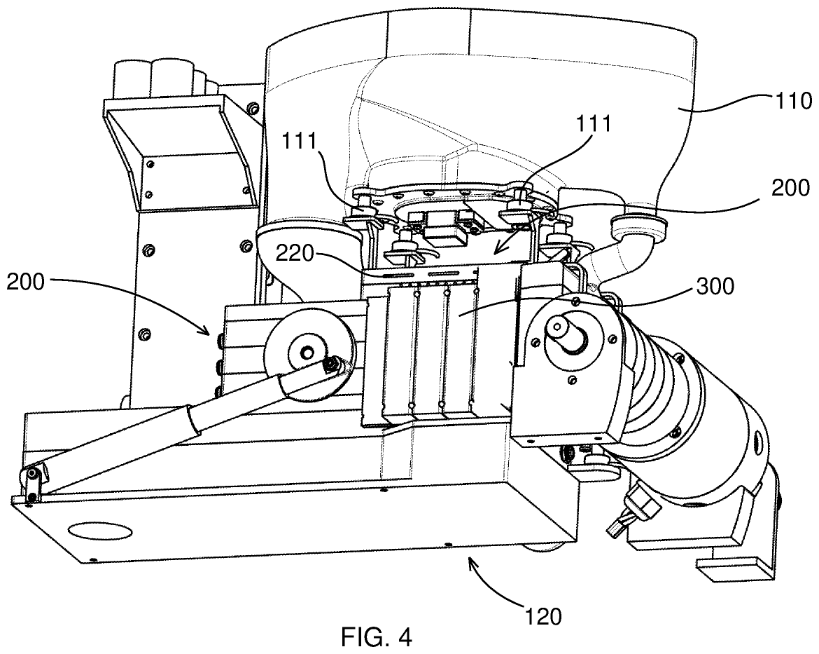

Now referring to FIG. 4, the interior of the toilet of FIGS. 1-3 is shown. The internal components of the toilet 100 are supported by a base 120. The bowl 110 is supported by one or more load cells 111. A manifold 200 is located below the bowl 110. The manifold 200 comprises a plurality of fluid paths. These fluid paths allow the manifold 200 to move fluids between the bowl 110, fluid containers 140, outside sources (e.g., municipal water supplies), other sources (e.g., air or water electrolyzing unit), analytical test devices 300, and the toilet outlet. The manifold 200 also provides electrical power and data connections to the analytical test devices 300. The manifold 200 can also directly pass fluids and/or solids from the bowl 110 to the toilet outlet. The manifold 200 provides multiple fluidic circuits including transport channels, valves, and pumps.

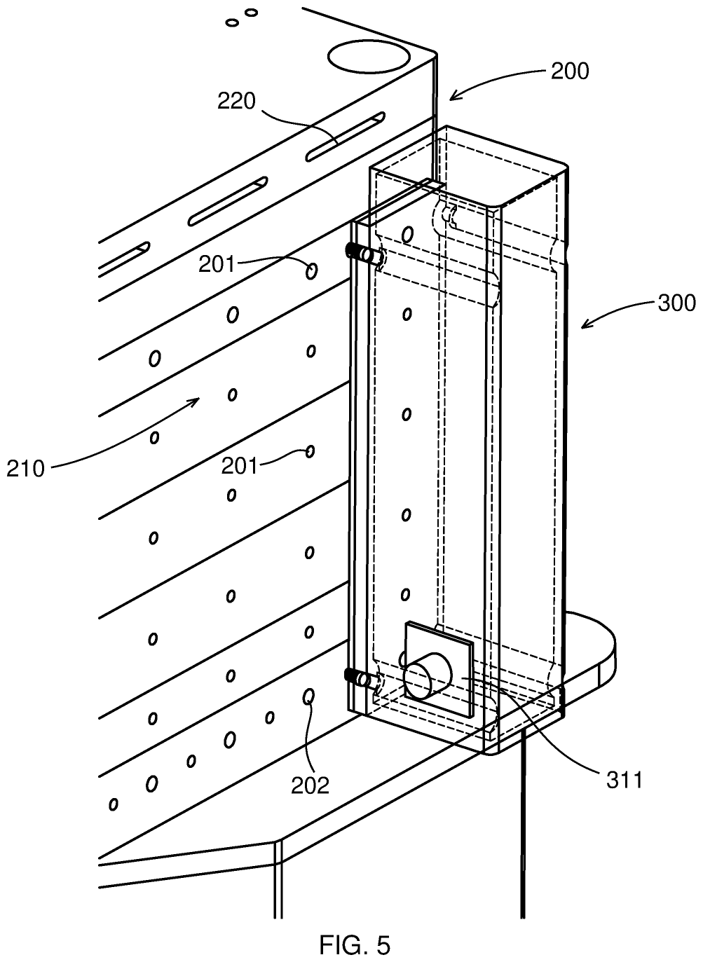

Now referring to FIG. 5, a first embodiment of a modular analytical test device 300 attached to an exemplary embodiment of a manifold 200 is shown. The manifold 200 is adapted to provide receptacles 210 with standardized connection interfaces for multiple analytical test devices 300. The manifold 200 is shown here with multiple fluid sources 201 for the analytical test device 300. In various embodiments, the manifold 200 may include receptacles 210 for more than one type of analytical test device 300 (e.g., different sizes, fluid supply needs, etc.).

In various exemplary embodiment, the analytical test device 300 includes multiple inlets in fluid communication with the manifold 200. The selected fluid flows are directed into a test chamber with one or more sensors 311 (flow channels internal to the analytical test device not shown in FIG. 5). The sensors 311 may be one or more of electrochemical sensors, spectrometers, chromatography, charge-coupled device (CCD), or metal oxide semiconductor field-effect transistor (MOSFET) including complementary metal oxide semiconductor field-effect transistor (CMOSFET). The analytic test device 300 also includes at least one outlet 302 or drain in fluid communication with the manifold 200.

In various exemplary embodiments, the analytic test device 300 includes one or more ports for a microfluidic chip (“MFC”). The microfluidic interfaces 220 are designed to receive a MFC and provide it with all necessary power, data, and fluidic connections. Microfluidics are used to transport samples and other fluids to and from the MFC. In a preferred embodiment, the MFC includes a test chamber with a lab-on-chip (“LoC”) (also known as test-on-chip). The LoC may be designed to perform one or more laboratory tests. The fluidic circuits and sensors may be as small as micro or nano sized.

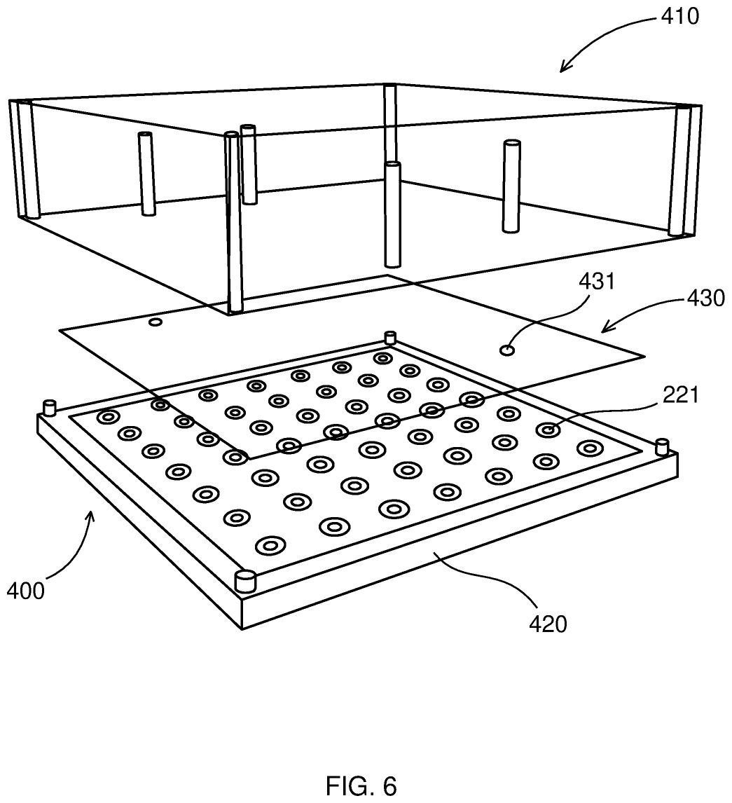

Now referring to FIG. 6, an exemplary embodiment of an analytical test device 300 adapted for use with a microfluidic chip (“MFC”) is shown. The MFC analytic test device 400 includes a test chamber 410 with a lab on chip 411. The manifold 200 includes a plurality of slots 220 or other openings for placement of a MFC analytical test device 400. An interface 420 with multiple ports 421, which act as fluid inlets or outlets for the test chamber 410, to provide connections and/or supplies for the MFC analytic test device 400. Protrusions 422 encircle the ports 421 to provide a point of positive contact for the sealing gasket 430, minimizing dead volume. Pores 431 in the gasket 430 select or block possible interactions with the MFC analytic test device 400.

In various exemplary embodiments, the backplate interface 420 is machined or molded with multiple microfluidic pores 421 for ingress of fluids or for removal of fluids. The interface 420 can be used with a variety of MFC analytic test device 400 testing modules. The ports 421 are preferably sealed by placing a gasket 430 between the MFC analytic test device 400 and backplate 420. The gasket 430 may be designed with pores 431 to selectively allow fluid flow through selected ports 421 and block potential flow through others depending on the MFC analytic test device 400 design. The gasket 430 may have alignment holes or features, with matching structures in the interface 420 or MFC analytic test device 400, to facilitate aligning the sheet of material to the pores 431. For example, the gasket 430 may fit snugly in a recess in the interface 420, or alignment pins in the interface 420 may match holes patterned in the gasket 430 by the same process used to create the pores 431. The gasket 430 may have corrugations, or variations in thickness, or be composed of multiple layers of different materials designed to reduce distortions propagating from one point of contact to another, in order to improve the alignment of the gasket 430 to the pores 431 during installation or when the system is pressurized.

In various exemplary embodiments, the gasket 430 may function as a seal for otherwise open channels in the MFC analytic test device 400, permitting pressurized flow in those channels. The gasket 430, typically an electrical insulator, may have electrical contacts built into the top, bottom, or intermediate layers of material, or embedded within the gasket 430 material. The gasket 430 may have electrical contacts built to cause a voltage potential to exist in the fluid. The gasket 430 may have electrical contacts built on the surface to come into contact with the fluid or gas and provide a potential to create electrochemical interactions. The gasket 430 may have electrical contacts built on the surface to come into contact with the fluid or gas and measure the electrical potential or ionic current.

In various exemplary embodiments, when the MFC analytic test device 400 is mechanically clamped to the plate 420 with sufficient pressure, the gasket 230 material creates a high-pressure seal between the MFC analytic test device 400 and the interface 420. Pores 431 in the gasket 430 open a channel between the backplate 420 and the MFC analytic test device 400. The gasket 430 material may be optimized for the application, including selecting chemically inert material. Alternatively, each pore 431 may be circumscribed with an elastomer O-ring that provides a seal under pressure.

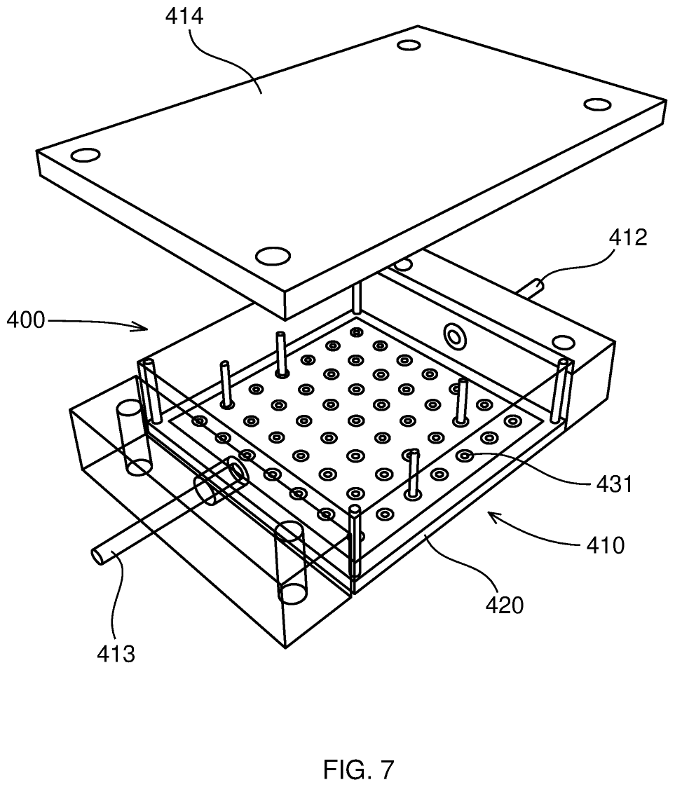

Now referring to FIG. 7, an exemplary embodiment of an MFC analytic test device 400 with an optical interface is shown. In some embodiments, a light source and light detector are connected to the test chamber 410 by fiber optic cables 412 and 413 are part of the test chamber 410 (e.g., spectrometer). The MFC analytic test device 400 is attached to the interface and held in place by a clamp plate. In an alternative embodiment, the light source 412 may be placed in the clamp plate 414 to deliver light to a standardized location normal to the MFC analytic test device 400.

In various exemplary embodiments, the light detector may include various mechanisms for reducing reflections, such as covering the detector with an anti-reflection coating, film. The light detector may be an optical waveguide or other photonic sink.

In various exemplary embodiments, the MFC analytic test device 400 is secured to the interface 420 by a clamp 414 In a preferred embodiment, the clamp 414 serves a dual purpose as a back-side interface for microfluidic ports 421 in the reverse side of the MFC analytic test device 400. Microchannels machined into the clamp 414 in standardized locations may be included in the clamp design.

In various exemplary embodiments, the microfluidic fixtures, including screw-in connectors, may interface with micro-tubing to provide a connection elsewhere in the system, or back to the same chip. The micro-tubing may provide chip-to-chip connections.

In various exemplary embodiments, the clamp may serve as a housing or mounting point for a mirror that directs laser light through the MFC analytic test device 400. Some implementations may use a semi-transparent mirror that allows several MFC analytic test devices 400 arranged in a line to use the same laser beam to interact with MFC analytic test device 400 components. The platform microfluidic interface/manifold provides ports intended for this purpose.

In various exemplary embodiments, the microfluidic fixtures, including screw-in connectors, may interface with micro-tubing to provide a connection elsewhere in the system, or back to the same chip. The micro-tubing may provide chip-to-chip connections.

In various exemplary embodiments, the clamp may serve as a housing or mounting point for a mirror that directs laser light through the MFC analytic test device 400. Some implementations may use a semi-transparent mirror that allows several MFC analytic test devices 400 arranged in a line to use the same laser beam to interact with MFC analytic test device 400 components. The platform microfluidic interface/manifold provides ports intended for this purpose.

In various exemplary embodiments, the MFC analytical test device 400 is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold provides routing. Interconnecting levels of channels allows routing from one port to all others. Each channel may include a pressure dampener to facilitate constant pressure pumping of all active channels simultaneously, while time-multiplexing pump-driven inflow. Fluids may be supplied by the manifold in continuous or segmented flow (e.g., separated by air bubbles). The sensors may collect data continuously during exposure or may take discrete data points. Once the testing parameters are reached and reliable data is collected that is within a pre-determined range, the data may then be statistically evaluated. The mean, median, and standard deviation of the data may be carried out. Additionally, regression analysis may be carried out on the data of a single user. Regression analysis may also be used on two or more users to understand how the data of a single user compares to a population of users of the analytical toilet.

In various exemplary embodiments, the manifold has reaction chambers built in for general purpose mixing operations. Each chamber has a macro-sized channel through which the manifold delivers a urine sample (filling the reaction chamber), and the chamber has a micro-sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, colorimeter, laser, UV, magnification).

In various exemplary embodiments, the sensor to detect a particular analyte is integrated into a planar substrate with the active portion of the sensor exposed. Example substrates may be glass, plastic, ceramic, metal, etc. The planar sensor may be affixed to the manifold as described for the semiconductor embodiment.

In various exemplary embodiments, the manifold interface has a matrix of ports, possibly laid out in a regular grid. These ports may be activated or closed via an external support manifold. Routing is fully programmable.

In various exemplary embodiments, the manifold directs one or more fluids to the analytical test device 300 or MFC analytical test device 400 to cleanse the devices. These may include cleaning solutions, disinfectants, and flushing fluids. In various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

Now referring to FIGS. 8 and 8A, an exemplary embodiment of an MFC analytical test device 400 is shown. The top of the MFC 400 is removed to show exemplary flow paths from the inlets to the test chamber. The MFC 400 preferably includes a plurality of inlet flow paths 440 for the test sample, a diluent (e.g., ionized water), and at least one reagent. The inlet flow paths 440 further comprise a variety of valves 441 (shown in more detail in FIG. 7A) that open and/or close sections of the inlet flow paths 440 altering the length of the actual flow path for a given fluid. The bypass valves 441 are designed to reduce dead volume in the flow paths 440. Adjusting the length of a given flow path 440 while using a constant pump pressure will alter the rate at which a fluid reached the test chamber 410 which in turn alters the ratio of fluids entering the test chamber 410.

In various exemplary embodiments, the circuit 400 may be part of one chip and the reaction chamber part of another. For example, fluids may be mixed in one chip that outputs a variable ratio flow to the next chip.

In various exemplary embodiments, the test chamber 410 includes or is part of an analytical tool, such as a spectrometer, that is used to analyze the contents of the test chamber. Depending on the purpose of the test, different reagents in different ratios of sample to diluent to reagent are required (e.g., detecting different analytes). The disclosed chip 400 with variable flow paths 440 allow for the provision of these fluids in a variety of ratios as appropriate for a given test and/or analyte. In various exemplary embodiment, different sections of the flow paths 440 have different dimensions (e.g., diameters) and/or different surface materials or treatments (e.g., hydrophilic or hydrophobic) that also affect the flow rate. In various exemplary embodiments, the bypass valves 441 are placed at the outlets which allows the circuit to be tuned to match impedance at the output.

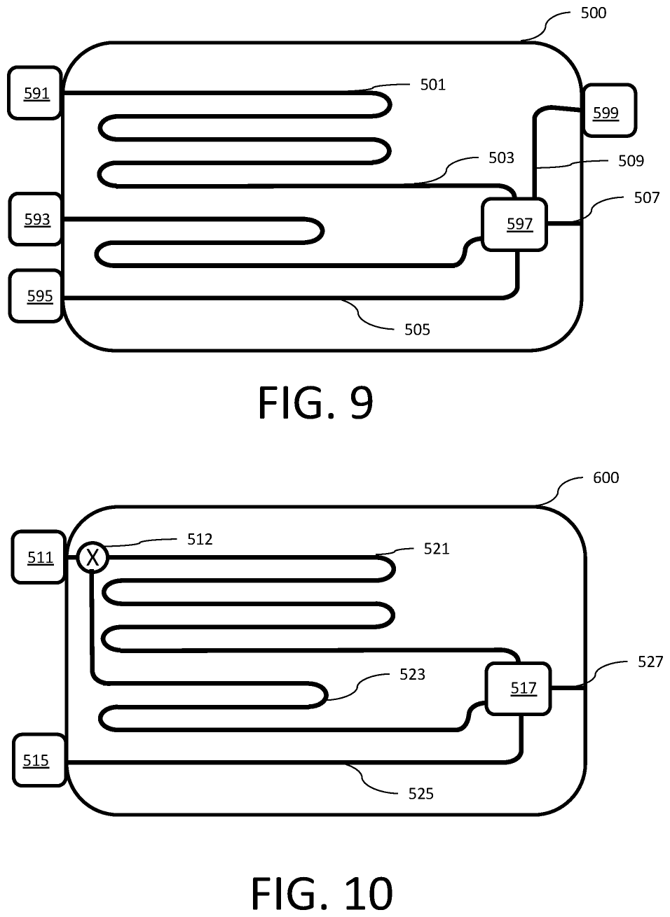

Referring to FIG. 9, a schematic view shows a microfluidic chip 500. A sensor, such as a photospectrometer, is represented as 597. A conduit bringing an excreta sample to the chip is represented as 591. A source of diluent, such as deionized water, is represented as 593. A source of a reagent is represented as 595. This embodiment also shows a source of wash liquid 599, which might also be deionized water. The wash liquid is carried to the sensor by path 509. Outlet 507 is provided for all the liquids to exit the sensor.

As seen in FIG. 9, the sample path 501 is longer than the diluent path 503 and the reagent path 505. Accordingly, assuming the same diameters of these paths, the sample path presents greater fluid resistance. As such, at the same pressure, there will be less sample delivered to the sensor 597 than the diluent or the reagent. Likewise, because the diluent path is longer than the reagent path, there will be less diluent delivered to the sensor than reagent. In accordance with the invention, these lengths can be changed to control the relative amounts of sample, reagent and diluent. For some analyses, it is desirable that more diluent is provided than reagent.

FIG. 10 is a schematic representation of another embodiment of a microfluidic chip 600. In this embodiment, a reagent source 515 is provided to deliver reagent to the sensor 517 through the reagent path 525. An outlet 527 is provided for the exit of liquids from the chip.

In FIG. 10, a conduit 511 is also provided to deliver sample to the chip 600. A valve 512 is provided to controllably select the first channel 523 or the second channel 521 for the sample path. The second channel 521 is preferably at least twice as long as the first channel. As such, when the second channel is selected by the valve, there is greater fluid resistance to the sample. Thus, assuming the same pressure, less sample is delivered to the sensor when the second channel 521 is selected. sample is delivered to the sensor 517 sample path. In alternative embodiments, the microfluidic chip is provided with more than 2 channels for the valve to select from, thus allowing even greater variation in the fluid resistance available on the chip.

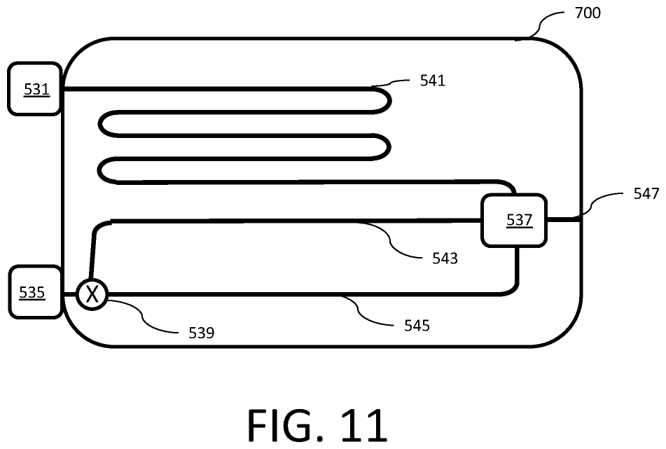

FIG. 11 represents an other embodiment of a microfluid chip 700. In the depicted embodiment, the conduit 531 brings sample to the chip. The sample is then carried to the sensor 537 on the sample path 541. Outlet 547 is provided for liquids to exit the chip.

In FIG. 11, a reagent source 535 provides a reagent to the chip. A controllable valve 539 is provided to select the first channel 543 or both the first channel 543 and the second channel 545 to carry the reagent to the sensor. When both the first and second channel are selected, the fluid resistance of the reagent is reduced. As such, and assuming the same pressure, more reagent is delivered to the sensor when it is allowed by the valve to travel through both channels.

In the embodiment shown in FIG. 10 and FIG. 11, the reagent in the reagent source may be switched out with another reagent, depending on what analysis is to be conducted. When this is done, it is particularly advantageous to vary the fluid resistance of either the sample or the reagent, to thereby tailor the delivery of both to fit the analyses.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl adapted to receive excreta;
a conduit for transporting a liquid excreta sample from the bowl;
a liquid reagent source; and
a microfluidic chip comprising:
a sensor configured to detect at least one property of the excreta sample;
an excreta sample path in fluid communication with the conduit and the sensor; and
a reagent path in fluid communication with the liquid reagent source and the sensor;
wherein the length of and number of channels in the sample path and the reagent path are selected so as to control the respective fluid resistance of the excreta sample and the reagent to thereby optimize the mixing and flow rates of the excreta sample and reagent into the sensor.

2. The analytical toilet of claim 1 further comprising a diluent source, wherein the microfluidic chip further comprises a diluent path in fluid communication with the dilutent source and the sensor, and wherein the length of and number of channels in the diluent path are selected so as to control the fluid resistance of the diluent to thereby optimize the mixing and flow rates of the diluent into the sensor.

3. The analytical toilet of claim 2 wherein the diluent comprises water.

4. The analytical toilet of claim 3 wherein the water comprises deionized water.

5. The analytical toilet of claim 1 further comprising a second liquid reagent source, and wherein the microfluidic chip further comprises a second reagent path in fluid communication with the second liquid reagent source and the sensor, and wherein the length of and number of channels in the second reagent path are selected so as to control the fluid resistance of the second reagent to thereby optimize the mixing and flow rates of the second reagent into the sensor.

6. The analytical toilet of claim 1, wherein the length of the sample path is at least twice as long as the reagent path, to thereby provide more reagent to the sensor than excreta sample under the sample pressure.

7. The analytical toilet of claim 1, wherein the reagent path comprises two or more channels, to thereby provide more reagent to the sensor than excreta sample under the same pressure.

8. The analytical toilet of claim 1, wherein the microfluidic chip further comprises an outlet in fluid communication with the sensor.

9. The analytical toilet of claim 1, wherein the reagent source and microfluidic chip are contained on a cartridge configured to fit into a receptacle in the toilet.

10. The analytical toilet of claim 1, wherein the sensor is selected from a spectrometer, a colorimeter and an image capture device.

11. An analytical toilet comprising:
a bowl adapted to receive excreta;
a conduit for transporting a liquid excreta sample from the bowl;
a liquid reagent source; and
a microfluidic chip comprising:
a sensor configured to detect at least one property of the excreta sample;
an excreta sample path in fluid communication with the conduit and the sensor; and
a reagent path in fluid communication with the liquid reagent source and the sensor, wherein the reagent path comprises a first and a second channel, wherein the second channel is longer than the first channel;
a valve, which valve is controllable so as to cause the reagent to flow through either the first channel, the second channel or both channels, to thereby control the fluid resistance of the reagent, to thereby optimize the flow rate of the reagent into the sensor.

12. The analytical toilet of claim 11, wherein the valve is controllable to cause the reagent to flow through either the first channel or the second channel, but not both, to thereby change the length of the reagent path and thereby increase the fluid resistance of resistance of the reagent path when the second channel is selected.

13. The analytical toilet of claim 11, wherein the valve is controllable to cause the reagent to flow through either the first channel or both the first and the second channel simultaneously, to thereby decrease the fluid resistance when the reagent flow through both the first and second channel simultaneously.

14. The analytical toilet of claim 11, the reagent source can be filled with either a first or a second reagent, depending on what analysis is to be performed, and wherein the valve is controlled depending on whether the first or the second reagent is in the reagent source.

15. The analytical toilet of claim 11, further comprising a second liquid reagent source, and wherein the microfluidic chip further comprises a second reagent path in fluid communication with the second liquid reagent source and the sensor.

16. The analytical toilet of claim 11 further comprising a diluent source, wherein the microfluidic chip further comprises a diluent path in fluid communication with the diluent source and the sensor.

17. The analytical toilet of claim 11, wherein the first channel is at least twice as long as the second channel.

18. The analytical toilet of claim 11, wherein the microfluidic chip further comprises an outlet in fluid communication with the sensor.

19. The analytical toilet of claim 11, wherein the reagent source and microfluidic chip are contained on a cartridge configured to fit into a receptacle in the toilet.

20. The analytical toilet of claim 11, wherein the sensor is selected from a spectrometer, a colorimeter and an image capture device.
